(19) United States
(12) Patent Application Publication
(10) Pub. No.: US 2026/0055045 A1
(43) Pub. Date: Feb. 26, 2026

(54) DOWNSTREAM SEPARATION OF MIXED DICARBOXYLIC ACIDS FROM OXIDATION OF HDPE VIA LIQUID-LIQUID EXTRACTION

(71) Applicant: Alliance for Sustainable Energy, LLC, Golden, CO (US)

(72) Inventors: Katrina Marie KNAUER, Denver, CO (US); Yuanzhe LIANG, Golden, CO (US)

(21) Appl. No.: 19/304,978

(22) Filed: Aug. 20, 2025

Related U.S. Application Data
(60) Provisional application No. 63/685,126, filed on Aug. 20, 2024.

(51) Int. Cl. C07C 51/48; B01D 11/04

(52) U.S. Cl. CPC C07C 51/48; B01D 11/0492

(57) ABSTRACT

Disclosed herein are methods for isolating dicarboxylic acids (DCAs) from about C₃-C₂₆ in carbon backbone length resulting from hydrothermal nitric acid oxidation, metal catalyzed oxidation or other oxidation means of polyethylene.

### CROSS-REFERENCE TO RELATED APPLICATIONS

This application claims priority under 35 U.S.C. § 119 to U.S. provisional patent application No. 63/685,126 filed on 20 Aug. 2024, the contents of which are hereby incorporated in their entirety.

### CONTRACTUAL ORIGIN

The United States Government has rights in this invention under Contract No. DE-AC36-08GO28308 between the United States Department of Energy and Alliance for Sustainable Energy, LLC, the Manager and Operator of the National Renewable Energy Laboratory. This invention was made under CRADA CRD-22-22451 between Proctor & Gamble and Alliance for Sustainable Energy, LLC. The United States Government has certain rights in this invention.

### BACKGROUND

Unlike mechanical recycling methods for plastic waste, alternative valorization techniques empower the conversion of polyolefins into functional building blocks. Through hydrothermal nitric acid oxidation or metal catalyzed oxidation, polyethylene (PE) waste undergoes deconstruction into a distribution of short-chain, value-added dicarboxylic acids (DCAs). These DCA's range from C3-C26 in chain length. These diacids can be used in several applications. For example, C5 diacids can be used to synthesize nylon polymers, C3-C22 mixtures can be used to synthesize polyols for polyurethane applications, C4-C7 and C10-C16 diacids can be converted into surfactants for use in personal care and cleaning product, and diacids with carbon numbers exceeding C13 can be effectively utilized to synthesize recyclable-by-design aliphatic polyester.

Despite these promising applications, the challenge remains in downstream separations of the DCAs for target applications. The complexity is further compounded by the heterogeneity of chain lengths, along with the similar physicochemical properties of diacids within comparable carbon number ranges. Thus, the achievement of fit-for-purpose recycling and upcycling of diacids from oxidized PE hinges on the precise isolation of diacid subgroups.

### SUMMARY

In an aspect, disclosed herein is a method for isolating a dicarboxylic acid derived from a mixture of oxidized polyethylene decomposition products wherein the isolated dicarboxylic acid consists of only one carbon backbone chain length; and wherein the method comprises at least one liquid-liquid extraction of the mixture of oxidized polyethylene decomposition products. In an embodiment, the liquid-liquid extraction comprises at least one solvent system. In an embodiment, the at least one solvent system comprises a mixture consisting of tetrahydrofuran, water and ethanol. In an embodiment, the at least one solvent system comprises a mixture of water and ethanol. In an embodiment, the at least one solvent system comprises a mixture of water and ethanol ranging from water (1−x) and ethanol (x) wherein x is within a range of from zero to 1. In an embodiment, the dicarboxylic acid has a carbon backbone chain length of from 3 to 22 carbons.

In an aspect, disclosed herein is a method for isolating a mixture of dicarboxylic acids derived from a mixture of oxidized polyethylene decomposition products wherein the isolated mixture of dicarboxylic acids comprise carbon backbone chain lengths varying from one to two backbone carbons; and wherein the method comprises at least one liquid-liquid extraction of the mixture of oxidized polyethylene decomposition products. In an embodiment, the liquid-liquid extraction comprises at least one solvent system. In an embodiment, the at least one solvent system comprises a mixture consisting of tetrahydrofuran, water and ethanol. In an embodiment, the at least one solvent system comprises a mixture of water and ethanol.

In an embodiment, the at least one solvent system comprises a mixture of water and ethanol ranging from water (1−x) and ethanol (x) wherein x is within a range of from zero to 1. In an embodiment, the dicarboxylic acid has a carbon backbone chain length of from 3 to 22 carbons.

Other objects, advantages, and novel features of the present invention will become apparent from the following detailed description of the invention when considered in conjunction with the accompanying drawings.

### BRIEF DESCRIPTION OF THE DRAWING

FIG. 1 depicts the solubility of DCAs having a carbon backbone chain length of from C3 to C17 in solutions of varying ratios of water to ethanol. In an embodiment, as depicted in FIG. 1, a mixture of diacids of different carbon numbers (n=7, 8, 10, 12, 13, 14, same mass for each diacid) were placed in 6 solvent systems comprising different ratios of water and ethanol (1, 8:2, 6:4, 4;6, 2:8, 0). After 3 hours of solubilization, aliquots were removed from the liquid phase and DCA solubility was measured via gas chromatography mass spectroscopy (GC-MS).

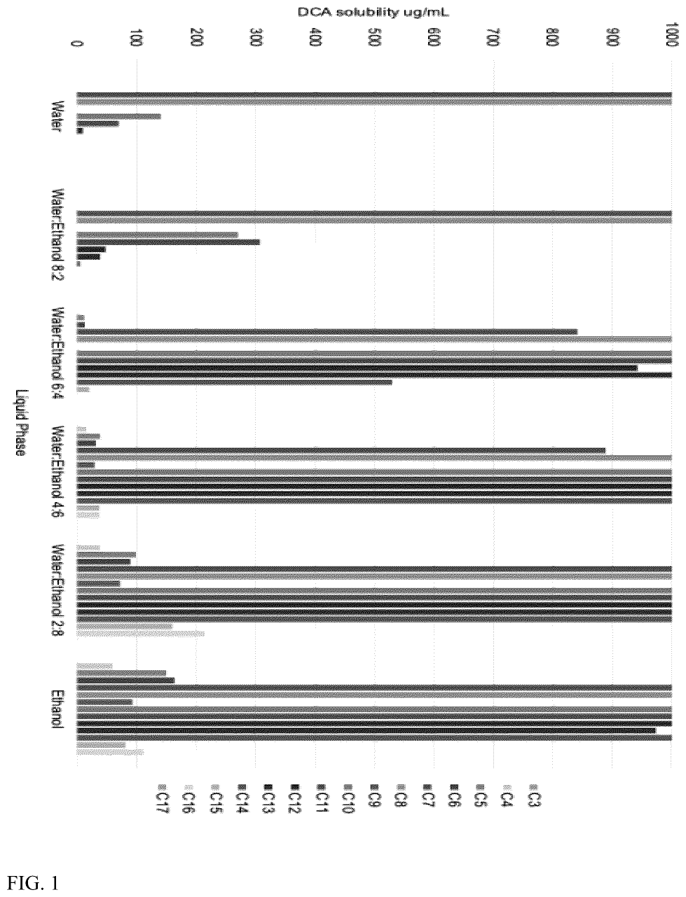

### DETAILED DESCRIPTION

Aerobic oxidation for polymer upcycling has several advantages including the ability of homogeneous catalysts to penetrate polymer networks more effectively than heterogeneous catalysts; tolerance to water and other contaminants; and the potential to use stable, recyclable catalysts. Methods are known for the oxidation of PE to dicarboxylic acids, but they lead to the formation of dicarboxylic acids of varying chain lengths with different properties.

Disclosed herein are novel compositions and methods useful in liquid-liquid extraction techniques for the separation of diacids resulting from the oxidative degradation of polyethylene. Previously, liquid-liquid extraction has been used for diacid recovery from diverse sources including leachates, fermentation broths, industrial wastes, and other streams within industrial processes. However, the emphasis was on the development of composite solvents incorporating nitrogen-based extractants, phosphorous-based extractants, and ionic liquids to achieve the extraction of a single diacid from a mixture. As a result, a regeneration step was necessary to restore the functionality of the extractants.

In contrast, using methods and compositions disclosed herein, one can isolate diacids into subgroups through liquid-liquid extraction based on their carbon backbone chain length. The isolated single carbon backbone length or mixture of diacids with varying carbon backbone lengths can be used for building blocks for synthesizing renewable polymers, lubricants, detergents, and more.

Accordingly, disclosed herein are methods for the separation and isolation of DCAs of varying chain lengths from a mixture of DCA products resulting from the oxidation of PE. The separation of DCAs is achieved through a sequential liquid-liquid extraction method utilizing diverse combinations of one or more solvent systems, such as water, ethanol, and tetrahydrofuran. Disclosed herein are methods for isolating C4-C22 diacids into distinct subgroups. This breakthrough enables the multipurpose recycling and upcycling of PE, marking a significant advancement in sustainable materials management.

### Example 1: Modeling Via COSMO-RS

In this example, the solubility of single DCAs in varying solvents was modeled using Conductor like Screening MOdel for Real Solvents (COSMO-RS). The results of the modeled DCA solubility in water, ethanol (EtOH), and THF are displayed in Table 1 where check marks indicate high solubility.

TABLE 1
Modeled solubility of varying DCAs in water, EtOH, and THF.

| | Water | EtOH | THF |
|---|---|---|---|
| C3 | X | X | X |
| C4 | X | X | X |
| C5 | X | X | X |
| C6 | X | X | X |
| C7 | X | X | X |
| C8 | | X | X |
| C10 | | X | X |
| C11 | | X | X |
| C12 | | X | X |
| C13 | | X | X |
| C14 | | | X |
| C15 | | | X |
| C16 | | | X |
| C17 | | | X |
| C18 | | | X |
| C19 | | | X |
| C20 | | | X |
| C21 | | | X |
| C22 | | | X |

### Example 2: Solubility of DCAs in Liquid Phases Based on Mass

Experimental procedure: A mixture of diacids of different carbon numbers (n=7, 8, 10, 12, 13, 14, same mass for each diacid) were placed in 6 solvent systems comprising different ratios of water and ethanol (1, 8:2, 6:4, 4;6, 2:8, 0). At equilibrium, the mass of each diacid in the liquid phase and solid phase was measured, respectively.

TABLE 2
Summary of mass (in mg) of DCAs in liquid and solid phases.
Separation Efficiency, Mass (liquid phase or solid phase)/
Mass (total) at equilibrium* 100%

| | | Pimelic | Suberic | Sebacic | Dodecanedioic | Tridecanedioic | Tetradecanedioic |
|---|---|---|---|---|---|---|---|
| Solvent | | Acid | Acid | Acid | Acid | Acid | Acid |
| System | Phase | (C7) | (C8) | (C10) | (C12) | (C13) | (C14) |
| Water | L | 93 | 13 | 1 | 0 | 0 | 0 |
| | S | 7 | 87 | 99 | 100 | 100 | 100 |
| Water:Ethanol | L | 89 | 18 | 2 | 0 | 2 | 0 |
| 8:2 | S | 11 | 82 | 98 | 100 | 98 | 100 |
| Water:Ethanol | L | 95 | 57 | 11 | 6 | 63 | 3 |
| 6:4 | S | 5 | 43 | 89 | 94 | 37 | 97 |
| Water:Ethanol | L | 95 | 96 | 67 | 93 | 95 | 30 |
| 4:6 | S | 5 | 4 | 33 | 7 | 5 | 70 |
| Water:Ethanol | L | 97 | 97 | 75 | 93 | 95 | 84 |
| 2:8 | S | 3 | 3 | 25 | 7 | 5 | 16 |
| Ethanol | L | 100 | 97 | 94 | 96 | 96 | 74 |
| | S | 0 | 3 | 6 | 4 | 4 | 26 |

Water proves to be an excellent solvent that enables separation of C7− (diacids with 7 and fewer carbons) from C7+.

A water/ethanol solvent system of 6:4 to 4:6 ratio represents a highly promising candidate to achieve separation of C13− and C13+.

These results indicate a sequential separation protocol for isolating a mixture of diacids into 3 subgroups (C3-C7, C8-C13, C14+).

### Example 3: Solubility Measurements of DCAs in Various Solvents Based on GC-MS

Experimental procedure: A mixture of diacids of different carbon numbers (n=7, 8, 10, 12, 13, 14, same mass for each diacid) were placed in 6 solvent systems comprising different ratios of water and ethanol (1, 8:2, 6:4, 4;6, 2:8, 0). After 3 hours of solubilization, aliquots were removed from the liquid phase and DCA solubility was measured via gas chromatography mass spectroscopy (GC-MS).

These results are similar to example 1 and indicate that water is an effective medium for selectively removing C7 and C8 diacids from a mixture. Ethanol can separate C10-C14 diacids. And ethanol:water mixtures can be tailored to select other carbon lengths as well.

The foregoing discussion and examples have been presented for purposes of illustration and description. The foregoing is not intended to limit the aspects, embodiments, or configurations to the form or forms disclosed herein. In the foregoing Detailed Description for example, various features of the aspects, embodiments, or configurations are grouped together in one or more embodiments, configurations, or aspects for the purpose of streamlining the disclosure. The features of the aspects, embodiments, or configurations, may be combined in alternate aspects, embodiments, or configurations other than those discussed above. This method of disclosure is not to be interpreted as reflecting an intention that the aspects, embodiments, or configurations require more features than are expressly recited in each claim. Rather, as the following claims reflect, inventive aspects lie in less than all features of a single foregoing disclosed embodiment, configuration, or aspect. While certain aspects of conventional technology have been discussed to facilitate disclosure of some embodiments of the present invention, the Applicants in no way disclaim these technical aspects, and it is contemplated that the claimed invention may encompass one or more of the conventional technical aspects discussed herein. The following claims are hereby incorporated into this Detailed Description, with each claim standing on its own as a separate aspect, embodiment, or configuration.

We claim:

1. A method for isolating a dicarboxylic acid derived from a mixture of oxidized polyethylene decomposition products wherein the isolated dicarboxylic acid consists of only one carbon backbone chain length; and wherein the method comprises at least one liquid-liquid extraction of the mixture of oxidized polyethylene decomposition products.

2. The method of claim 1 wherein the liquid-liquid extraction comprises at least one solvent system.

3. The method of claim 2 wherein the at least one solvent system comprises a mixture consisting of tetrahydrofuran, water and ethanol.

4. The method of claim 2 wherein the at least one solvent system comprises a mixture of water and ethanol.

5. The method of claim 2 wherein the at least one solvent system comprises a mixture of water and ethanol ranging from water (1−x) and ethanol (x) wherein x is within a range of from zero to 1.

6. The method of claim 2 wherein the at least one solvent system comprises a mixture of water and ethanol consisting of molar ratios of water to ethanol of 8 to 2, 6 to 4, 4 to 6 and 2 to 8.

7. The method of claim 1 wherein the dicarboxylic acid has a carbon backbone chain length of from 3 to 22 carbons.

8. The method of claim 1 wherein the dicarboxylic acid has a carbon backbone chain length of from 3 to 17 carbons.

9. The method of claim 1 wherein the dicarboxylic acid has a carbon backbone chain length of from 7 to 14 carbons.

10. The method of claim 1 wherein the dicarboxylic acid is selected from the group consisting of pimelic acid, suberic acid, sebacic acid, dodecanedioic acid, tridecanedioic acid, and tetradecanedioic acid.

11. A method for isolating a mixture of dicarboxylic acids derived from a mixture of oxidized polyethylene decomposition products wherein the isolated mixture of dicarboxylic acids comprise carbon backbone chain lengths from 3 to 22 backbone carbons; and wherein the method comprises at least one liquid-liquid extraction of the mixture of oxidized polyethylene decomposition products.

12. The method of claim 11 wherein the liquid-liquid extraction comprises at least one solvent system.

13. The method of claim 12 wherein the at least one solvent system comprises a mixture consisting of tetrahydrofuran, water and ethanol.

14. The method of claim 12 wherein the at least one solvent system comprises a mixture of water and ethanol.

15. The method of claim 12 wherein the at least one solvent system comprises a mixture of water and ethanol ranging from water (1−x) and ethanol (x) wherein x is within a range of from zero to 1.

16. The method of claim 12 wherein the at least one solvent system comprises a mixture of water and ethanol consisting of molar ratios of water to ethanol of 8 to 2, 6 to 4, 4 to 6 and 2 to 8.

17. The method of claim 11 wherein the dicarboxylic acids comprise carbon backbone chain lengths of from 3 to 17 carbons.

18. The method of claim 11 wherein the dicarboxylic acids comprise carbon backbone chain lengths of from 7 to 14 carbons.

19. A method for isolating a mixture of dicarboxylic acids derived from a mixture of oxidized polyethylene decomposition products wherein the isolated mixture of dicarboxylic acids comprise carbon backbone chain lengths selected from the group consisting of from 3 to 7, from 8 to 13 and from 14 to 22 backbone carbons; and wherein the method comprises at least one liquid-liquid extraction of the mixture of oxidized polyethylene decomposition products; and wherein the liquid-liquid extraction solution is selected from the group consisting of molar ratios of water to ethanol of 1 to 0, 8 to 2, 6 to 4, 4 to 6, 2 to 8, and 0 to 1.

20. The method of claim 19 wherein the solvent system comprises THF.
